(19) United States
(12) Patent Application Publication
(10) Pub. No.: US 2017/0232159 A1
(43) Pub. Date: Aug. 17, 2017

(54) INFANT NASAL SUCTION APPARATUS

(71) Applicant: Jessica Mackey-Jean, Brooklyn, NY (US)

(72) Inventor: Jessica Mackey-Jean, Brooklyn, NY (US)

(21) Appl. No.: 15/044,271

(22) Filed: Feb. 16, 2016

(51) Int. Cl. A61M 1/00

(52) U.S. Cl. CPC A61M 1/0003; A61M 1/008; A61M 2205/3365; A61M 2205/8206

(57) ABSTRACT

The infant nasal suction apparatus is disclosed for clearing the nasal passages of small babies and toddlers. The invention provides stress-free mucus extraction and allows the child to breathe easily and freely. The nasal suction apparatus is comprised of the suction housing, which includes the motor, battery, suction level adjustment controls, and an on/off switch. A collection container is removably connected to the suction housing, and a fluid conduit is connected to the container and adapted for placement in a nostril. The infant nasal suction apparatus is arranged for the removal of excess fluid and mucus from an infant nasal passageway and is controlled by the provided speed adjustment controller.

### CROSS REFERENCE TO RELATED APPLICATION

Not Applicable

### FEDERALLY SPONSORED RESEARCH AND DEVELOPMENT

Not Applicable

### MICROFICHE APPENDIX

Not Applicable

### BACKGROUND OF THE INVENTION

(1) Field of Invention

The present invention relates to a nasal apparatus, and, more particularly, pertains to a new and improved infant nasal suction apparatus wherein the same is arranged for the extraction of mucus and the like from an infant's nasal passageways.

(2) Background of Invention

Infants require assistance with clearing nasal congestion until they acquire the ability to blow their noses. Since colds and flu are very common among infants, parents find themselves regularly clearing their children's noses.

Aspirators for clearing congestion in infants are found in hospitals and in the domestic environment. Aspirators used in hospitals are generally electric machines that, while effective, are too expensive for application in the household. Less expensive domestic aspirators are usually hand-held and ‘pear’ shaped with a pump. One of the dangers with these is that since they lack a one-way valve, there is the possibility that the congestion will be pushed further up, the nasal cavity rather than being sucked out.

Such a device is exemplified by Rocco J. DeLeonardis in U.S. Pat. No. 5,800,425, which provides an “Automatic Nasal Apparatus” for cleaning matter in the nasal canal of an infant. This device is operated by a motor, which imparts a suction action to the collection member through the hose, wherein the suction action causes the matter to be cleaned from the nasal canal and collected in the collection member.

U.S. Pat. No. 5,342,329, issued to Raul J. Croquevielle, discloses a portable, closed circuit, disposable device for post operatory surgical suction. The circuit is comprised of a bellows-shaped body receptacle for suctioned fluids, a threaded neck located on the upper surface of the bellows-shaped body that is suitable to receive a threaded cap, a cap provided with a suction tube system connected through the cap into the bellows-shaped body, and a check-valve for the system of tubes. However, this device requires manual pumping in order to operate.

U.S. Pat. No. 6,263,875, issued to Emory and Teata Pace, provides a child nasal decongesting device. This device includes a bulb defining an air cavity therein and a facemask outwardly extending from the bulb. The facemask has an open distal end opposite the bulb, which is designed for positioning over a mouth and nostrils of a user such that the mouth and nostrils of the user are covered by the distal end of the facemask.

Angus S. Filshie's U.S. Pat. No. 5,318,548 discloses an extractor that is particularly useful for extracting mucus from the nasal passages of new born babies. It is comprised of a manually operable hand pump with a separate compartment for the extracted mucus. This device also has the disadvantage that it must be manually pumped in order to operate.

The prior art devices are either difficult and tiring to use in the case of the manually powered suction devices, or are too complex and require many costly parts in the case of the battery powered devices. Thus, a need exist for a portable, battery powered suction device that is simple to manufacture and use and is economical.

### BRIEF SUMMARY OF THE INVENTION

In view of the foregoing disadvantages inherent in the known types of suction devices now present in the prior art, the present invention provides a new and improved infant nasal suction apparatus, wherein the same provides a portable battery powered suction device for effectively cleaning the nasal canal and a process to use the various components employed in the procedure. As such, the general purpose of the present invention, which will be described subsequently in greater detail, is to provide a new and improved infant nasal suction apparatus that has all of the advantages of the prior art, and none of the disadvantages.

Accordingly, it is a primary objective of the present invention to provide an infant nasal suction apparatus for clearing nasal passages.

In another aspect, the present invention provides a nasal suction apparatus specifically designed for small babies and toddlers that provides stress-free mucus extraction and allows them to breathe easily and freely.

It is yet another aspect of the present invention where the infant nasal suction apparatus is provided with a speed adjustment controller, through which the suction level can be adjusted.

Still another aspect of the present invention provides an infant nasal suction apparatus that is arranged for the removal of excess fluid and mucus from an infant's nasal passageway.

A further aspect of the present invention provides an infant nasal suction apparatus that is simple in construction and easy to use.

It is another objective of the present invention to provide a new and improved infant nasal suction apparatus that may be easily and efficiently manufactured and marketed.

It is a further aspect of the present invention to provide a new and improved infant nasal suction apparatus that is of a durable and reliable construction.

An even further objective of the present invention is to provide a new and improved infant nasal suction apparatus that is cheap to manufacture with regard to both materials and labor, and that, accordingly, is then susceptible of low prices of sale to the consuming public, thereby making such infant nasal suction apparatus economically available to the buying public.

Yet another objective of the present invention is to provide a new and improved infant nasal suction apparatus that incorporates, in the apparatuses and methods of the prior art, some of the advantages thereof, while simultaneously overcoming some of the disadvantages normally associated therewith.

Other aspects of the present invention will become apparent, from time to time, throughout the specification, as hereinafter related.

In this respect, before explaining at least one embodiment of the invention in detail, it is to be understood that the invention is not limited in its application to the details of construction and to the arrangements of the components set forth in the following description or illustrated in the drawings. The invention is capable of other embodiments, and of being practiced and carried out in various ways. Also, it is to be understood that the phraseology and terminology employed herein are for the purpose of description, and should not be regarded as limiting.

These, together with other objectives of the invention and the various features of novelty that characterize the invention, are pointed out with particularity in the disclosure. For a better understanding of the invention, its operating advantages, and the specific objectives attained by its uses, reference should be had to the accompanying drawings and descriptive matter, in which there are illustrated preferred embodiments of the invention.

### BRIEF DESCRIPTION OF THE DRAWINGS

To further clarify various aspects of some example embodiments of the present invention, a more particular description of the invention will be rendered by reference to specific embodiments thereof, which are illustrated in the appended drawing. It is appreciated that the drawing depicts only illustrated embodiments of the invention, and is, therefore, not to be considered limiting of its scope. The invention will be described and explained with additional specificity and detail through the use of the accompanying drawing, in which:

FIG. 1 is a perspective view of the infant nasal suction apparatus.

### DETAILED DESCRIPTION OF THE INVENTION

Various aspects of the illustrative embodiments will be described using the terms commonly employed by those skilled in the art to convey the substance of their work to others skilled in the art. However, it will be apparent to those skilled in the art that the present invention may be practiced with only some of the described aspects. For purposes of explanation, specific numbers, materials, and configurations are set forth in order to provide a thorough understanding of the illustrative embodiments. However, it will be apparent to one skilled in the art that the present invention may be practiced without the specific details. In other instances, well-known features are omitted or simplified in order to not obscure the illustrative embodiments.

In some preferred embodiments, the present invention provides an infant nasal suction apparatus for clearing the nasal passages of small babies and toddlers. The invention provides stress-free mucus extraction and allows them to breathe easily and freely. The apparatus is arranged for the removal of excess fluid and mucus from an infant nasal passageway. For this purpose, the nasal suction apparatus is equipped with the rubber suction tube, through which the mucus travels to the disposal container. The invention includes a speed adjustment controller, for adjusting the suction level. The nasal suction apparatus is powered by the rechargeable battery.

In accordance with the invention, the nasal suction apparatus includes a suction housing, a disposable collection container connected to the suction housing, and a fluid conduit suction tube connected to the housing and adapted for placement in a nostril. Together, the suction housing, container, and conduit define an air path. The suction housing encloses a battery and a motor to pulsate it. The suction housing also includes speed adjustment buttons for controlling and adjusting the speed for nasal suction. The open end of the collection container includes prominent edges for attachment with the suction housing. The fluid conduit includes a mucus tube for placement into a nostril. Pulsation, when generated, moves air through the discharge passage and generates vacuum pressure in the fluid conduit to draw mucus into the fluid conduit.

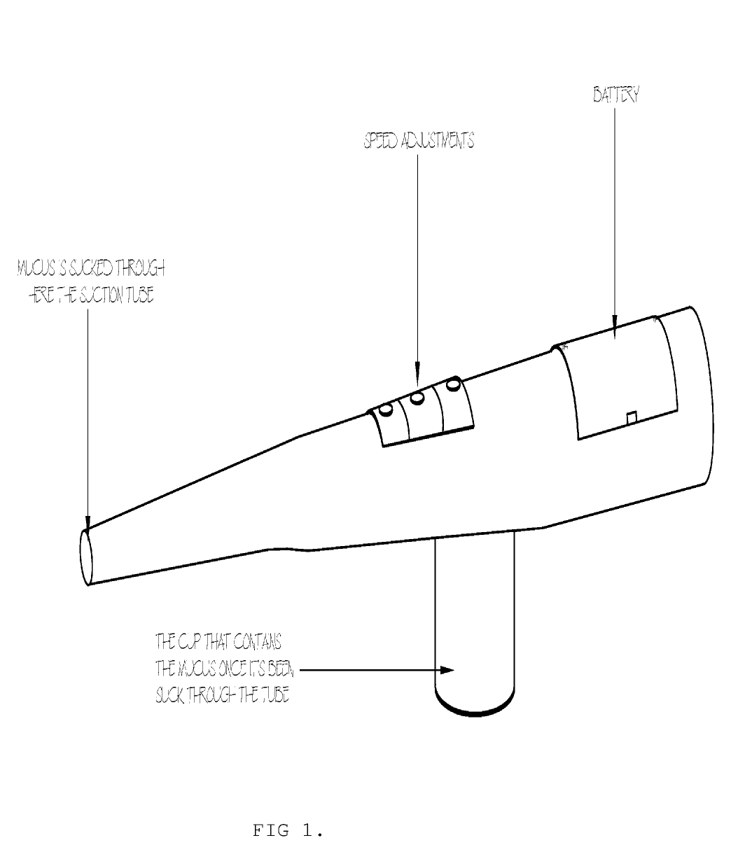

With reference now to the drawings, and in particular to FIG. 1 thereof, a new and improved nasal suction apparatus embodying the principles and concepts of the present invention is described herein. An embodiment of the nasal suction apparatus according to this invention is designated generally by the reference character 100.

The nasal suction apparatus 100 of this invention is comprised of a suction housing 10, a collection container 20 removably connected to the suction housing 10, and a fluid conduit suction tube 30 that is removably connected to the suction housing 10.

The suction housing 10 includes a motor that is connected to a battery or other DC source (not shown) enclosed in the compartment 11, and speed adjustment controls 12 and an on-off switch 14 mounted on an outside wall of the suction housing 10.

The U-shaped collection container 20 includes transparent sidewalls 21 with the open end 22 removably connected to suction housing 10 and a closed bottom end 23. The collection container 20 is transparent to see the amount of mucus collected and thereby disposed after being filled.

The conduit 30 includes a flexible tube 31 with a tapered probe or tip 32 on its end for placement into the nostrils for the removal of mucus. The fluid conduit suction tube 30 is preferably made of rubber and safe enough to insert in the nostrils of a child.

The lightweight design and plastic composition of the nasal suction apparatus 100 of the present invention is sturdy, and very easy to use and clean. Completely safe and comfortable for the child, the tapered probe or tip of the fluid conduit fits against the child's nostril without entering the nasal canal, thereby rendering the suctioning endeavor less terrifying for the child. As such, the clearing of the nasal passages can be completed in an expedient and easy manner. By keeping a child's nose clear of bacteria and mucus, further infection and illness can be prevented, a positive step for both the parent and the child.

Furthermore, the nasal suction apparatus can also help care for elderly and disabled persons, with larger devices employed for these patients who are unable to clear their nasal passages on their own.

As may now be appreciated from the above description and reference to the accompanying drawings, the present invention provides a nasal suction apparatus that is a clean, safe, and effective way to help young children clear a stuffy nose. Most importantly, such efficient clearing of the nose will help prevent further infection, discomfort, and perhaps a trip to the doctor. Fully developed, the nasal suction apparatus will be received positively by the vast majority of parents with young children.

It is to be understood that the above description is intended to be illustrative, and not restrictive. For example, the above-discussed embodiments may be used in combination with each other. Many other embodiments will be apparent to those of skill in the art upon reviewing the above description.

With respect to the above description then, it is to be realized that the optimum dimensional relationships for the parts of the invention, to include variations in size, materials, shape, form, function and manner of operation, assembly, and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Furthermore, since numerous modifications and changes will readily occur to those skilled in the art, it is not desired to limit the invention to the exact construction and operation shown and described, and, accordingly, all suitable modifications and equivalents may be resorted to, falling within the scope of the invention.

What is claimed is:

1. An infant nasal suction apparatus comprising:
suction housing, a collection container removably connected to said suction housing, and a fluid conduit connected to said container and adapted for placement in a nostril, said suction housing, container, and conduit together defining an air path;
said suction housing includes a motor, battery, suction level adjustment controls, and an on/off switch;
said fluid conduit including a mucus tube that is adapted for placement into a nostril;
whereby said air path is pulsated to move air through said discharge passage and generate vacuum pressure in said fluid conduit to thereby draw mucus from nostrils.

2. The infant nasal suction apparatus as claimed in claim 1, wherein said suction apparatus is utilized for clearing nasal passages specifically for small babies and toddlers.

3. The infant nasal suction apparatus as claimed in claim 1, wherein said suction apparatus provides stress-free mucus extraction and allows the child to breathe easily and freely.

4. The infant nasal suction apparatus as claimed in claim 1, wherein said suction apparatus is arranged for the removal of excess fluid and mucus from an infant nasal passageway.

5. The infant nasal suction apparatus as claimed in claim 1, wherein the suction level is adjusted through said speed adjustment controller.

6. The infant nasal suction apparatus as claimed in claim 1, wherein said suction apparatus is operated by battery.
